SEQUENCE LISTING (12 sequences)

SEQ ID NO: 1 - protein, 412 aa, Pasteurella multocida
MKNRRLNFKLFFLIIFSLFSTLSWSKTITLYLDPASLPALNQLMDFTQNNEDKTHPRIFGLSRFKIPDNIITQYQNIHFVELKDNRPTEALFTILDQYPGNIELDIHLNIAHSVQLIRPILAYRFKHLDRVSIQRLNLYDDGSMEYVDLEKEENKDISAEIKQAEKQLSHYLLTGKIKFDNPTIARYVWQSAFPVKYHFLSTDYFEKAEFLQPLKEYLAENYQKMDWTAYQQLTPEQQAFYLTLVGFNDEVKQSLEVQQAKFIFTGTTTWEGNTDVREYYAQQQLNLLNHFTQAGGDLFIGDHYKIYFKGHPRGGEINDYILNNAKNITNIPANISFEVLMMTGLLPDKVGGVASSLYFSLPKEKISHIIFTSNKQVKSKEDALNNPYVKVMRRLGIIDESQVIFWDSLKQL

SEQ ID NO: 2 - DNA, 1236 bp, Pasteurella multocida
atgaaaaatcgtcgactcaatttcaaactgtttttcctcatcattttttcattattcagcacactgagttggtcaaaaacaatcacgctgtatttagatcctgcctccttaccggcattaaatcagctgatggactttacgcaaaataatgaagataaaacacatccacgtatttttggtctttctcgctttaaaatccctgacaacattattacacagtatcaaaatatccatttcgtcgaactcaaagataatcgtcccactgaagcactttttacgattttggatcaataccctggtaacattgagttagatatacacttaaatattgctcattccgttcaattaattcgtccgattttggcatatcgttttaaacatttagatcgtgtatcaattcagcgattaaatctttatgacgatggctcaatggaatatgttgatttagaaaaagaagaaaataaagatatttccgcagaaattaagcaagcagaaaaacaactttctcactatttgcttaccggtaaaataaaatttgataacccaactattgctcgttatgtctggcaatccgcgttcccagtaaaatatcattttttaagtacagactattttgaaaaagccgaatttttacaaccactaaaagaatatttagcggaaaattatcaaaaaatggactggaccgcttaccaacagctgactccagaacagcaagcattctacttaacattggtaggcttcaatgacgaagtcaagcagtcgctagaagtgcaacaagccaaatttatctttaccggcacgacaacttgggaaggaaataccgatgtgcgagaatactacgcacagcaacaacttaatttacttaatcactttacccaagctgggggcgatttatttattggtgatcattataaaatctactttaaagggcatcctagaggtggtgaaattaatgactacattctgaacaatgctaaaaatatcaccaatatccctgccaatatttcctttgaagtattgatgatgacaggcttattacctgataaagtgggtggtgttgcaagttcactgtatttctccttaccaaaagaaaaaattagccatattattttcacatcgaataaacaagtgaaaagcaaagaagatgcgctaaataatccgtatgttaaggtcatgcgtcgtttaggtataattgacgaatcacaagtcatcttttgggacagtttaaaacagttg

SEQ ID NO: 3 - protein, 308 aa, Pasteurella multocida
MNLIICCTPLQVLIAEKIIAKFPHMPFYGVMLSTVSNKKFDFYAKRLAQQCQGFFSMVQHKDRFNLLKEILYLKRTFSGKHFDQVFVANINDLQIQFLLSAIDFNLLNTFDDGTINIVPNSLFYQDDPATLQRKLINVLLGNKYSIQSLRALSHTHYTIYKGFKNIIERVEPIELVAADNSEKVTSAVINVLLGQPVFAEDERNIALAERVIKQFNIHYYLPHPREKYRLAQVNYIDTELIFEDYILQQCQTHKYCVYTYFSSAIINIMNKSDNIEVVALKIDTENPAYDACYDLFDELGVNVIDIRE

SEQ ID NO: 4 - DNA, 924 bp, Pasteurella multocida
atgaatttgattatttgttgtacaccgttacaggtgttgattgcagaaaaaattatcgctaaatttccgcatatgccattttatggcgtcatgctttcaacagtcagtaataaaaaatttgatttttatgcaaagcggcttgcgcaacagtgccaaggttttttttccatggtgcagcataaggatcgcttcaatctattaaaagaaattctgtatttaaaacgaacattttcgggtaagcactttgatcaggtttttgtggcaaacattaatgacttacaaattcagtttttattaagtgccatcgactttaatctgttaaatacctttgatgacggcacaattaatattgtaccgaatagtcttttttaccaagatgaccctgccacgttgcagcgaaaactgattaatgtgctgttaggtaataaatacagtattcaatcattacgcgctttatcccatacacactacactatttataaaggcttcaagaatattattgaacgggtagagccgattgaattggtcgcagcagataacagtgaaaaagtcacttcagcggtgattaacgtattgcttgggcaacccgtttttgctgaagatgaacgcaatattgccttagcggaacgcgtgatcaaacaatttaatattcattattatttgcctcatccacgcgaaaagtatcgtttagcccaagtcaattacattgatacggaattgatctttgaagattatattcttcagcaatgtcaaacccacaaatactgtgtttatacatattttagtagcgccattattaatatcatgaataaaagtgacaatattgaagtggtagcattaaaaattgacacagaaaatcccgcctatgatgcttgttatgatttgtttgatgagctaggcgttaacgttattgatataagagag

SEQ ID NO: 5 - protein, 303 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLGTIVSKKSRFHSNLIVRLIRDLLKLPSALKHYLKEK

SEQ ID NO: 6 - DNA, 909 bp, Pasteurella multocida
atggataagttcgcagaacatgaaataccgaaagccgttattgttgcagggaatggcgagagtttaagtcaaattgattataggttgttaccgaaaaattatgatgtgtttcgttgtaatcaattttattttgaagaacgctattttttaggaaacaagataaaagcagttttcttcacgccaggggtctttcttgagcaatattatacactttatcatctcaagagaaacaatgagtattttgttgataatgtgattctctcttcttttaatcatcctacagtagatttagaaaagagtcagaaaatacaagcactttttattgatgtgatcaacggatatgaaaagtatttatctaaactcactgcttttgatgtttatttgcgctataaagaattatatgagaatcaaagaattacatctggcgtatatatgtgtgcagttgctattgcgatgggatatacagatatttacttaactggtatcgatttttatcaagcgagcgaagaaaactacgcattcgataataaaaagcctaacattattaggttattgcctgattttcggaaagaaaaaacactcttttcttatcatagtaaagatattgatttggaagcattatcttttttacaacagcattatcatgttaatttttattcaatttcaccaatgagccctttgtctaaacattttcctattccaactgtagaggatgattgtgaaacaacttttgttgcgccactaaaagaaaattacattaatgatatattgttgcctcctcattttgtatatgaaaaattagggaccatcgtgtctaagaaatcacgttttcattctaacttgattgtcaggttgattagagacttattgaaattaccgagtgcacttaaacactatttaaaagaaaaa

SEQ ID NO: 7 - DNA, 29 bp, Artificial Sequence
  Description of Artificial SequenceSynthetic DNA
atcatcgatatgaaaaatcgtcgactcaa

SEQ ID NO: 8 - DNA, 29 bp, Artificial Sequence
  Description of Artificial SequenceSynthetic DNA
tatggatccttacaactgttttaaactgt

SEQ ID NO: 9 - DNA, 25 bp, Artificial Sequence
  Description of Artificial SequenceSynthetic DNA
gtaatcgatgacggctaaaagtagg

SEQ ID NO: 10 - DNA, 32 bp, Artificial Sequence
  Description of Artificial SequenceSynthetic DNA
ttcggatccttactctcttatatcaataacgt

SEQ ID NO: 11 - DNA, 29 bp, Artificial Sequence
  Description of Artificial SequenceSynthetic DNA
ggaatcgatatggataagttcgcagaaca

SEQ ID NO: 12 - DNA, 29 bp, Artificial Sequence
  Description of Artificial SequenceSynthetic DNA
taaggatccttatttttcttttaaatagt

(19) United States
(12) Patent Application Publication
(10) Pub. No.: US 2005/0089956 A1
(43) Pub. Date: Apr. 28, 2005

(54) Process for producing alpha 2,3/ alpha 2,8-sialyltransferase and sialic acid-containing complex sugar

(75) Inventors: Tetsuo Endo, Palo Alto, CA (US); Satoshi Koizumi, Yokohama (JP)

Correspondence Address: FITZPATRICK CELLA HARPER & SCINTO, 30 ROCKEFELLER PLAZA, NEW YORK, NY, 10112, US

(73) Assignee: Kyowa Hakko Kogyo Co. Ltd., Tokyo (JP)

(21) Appl. No.: 10/491,067

(22) PCT Filed: Sep. 26, 2002
(86) PCT No.: PCT/JP02/09907

(30) Foreign Application Priority Data
Sep. 26, 2001 (JP) 2001-292796

(51) Int. Cl. C12N 9/10; C07H 21/04; C12N 1/21; C12N 15/74

(52) U.S. Cl. 435/69.1; 435/193; 435/252.3; 435/320.1; 536/23.2

(57) ABSTRACT

The present invention can provide a process for producing a protein having α2,3/α2,8-sialyltransferase activity using a transformant comprising a DNA encoding a protein having α2,3/α2,8-sialyltransferase activity derived from a microorganism belonging to the genus Pasteurella and a process for producing a sialic acid-containing complex carbohydrate using a transformant capable of producing a protein having α2,3/α2,8-sialyltransferase activity derived from a microorganism.

### TECHNICAL FIELD

The present invention relates to a process for producing a protein having α2,3/α2,8-sialyltransferase activity using a transformant comprising a DNA encoding a protein having α2,3/α2,8-sialyltransferase activity and a process for producing a sialic acid-containing complex carbohydrate.

### BACKGROUND ART

As α2,3-sialyltransferase and its genes, genes derived from animals [J. Biol. Chem., 267, 21011 (1992), J. Biol. Chem., 268, 22782 (1993), Eur. J. Biochem., 216, 377 (1993), Biochem. Biophys. Res. Commun., 194, 375 (1993), J. Biol. Chem., 269, 1394 (1994), J. Biol. Chem., 269, 10028 (1994), Glycobiology, 5, 319 (1995)] and the like have been obtained. Although there is an example in which the gene encoding the α2,3-sialyltransferase derived from an animal was expressed in bacteria such as Escherichia coli and a protein having activity was obtained (Japanese Published Unexamined Patent Application No. 2531631/99), its activity was very weak. Also, α2,8-sialyltransferase and its genes, genes derived from animals [Proc. Natl. Acad. Sci. USA, 91, 7952 (1994), Proc. Natl. Acad. Sci. USA, 91, 10455 (1994), J. Biol. Chem., 269, 15950 (1994), J. Biol. Chem., 271, 3684 (1996), J. Biol. Chem., 270, 14628 (1995)] and the like have been obtained. However, there is no example in which the gene encoding the α2,8-sialyltransferase derived from an animal was expressed in bacteria such as Escherichia coli and a protein having activity was obtained.

On the other hand, in the case of microorganisms, there is a report in which a gene encoding the α2,3-sialyltransferase was obtained from microorganisms belonging to the genus Neisseria, the genus Campylobacter and the genus Haemophilus and the α2,3-sialyltransferase was expressed in Escherichia coli using the gene [U.S. Pat. No. 6,096,529, WO97/47749, WO99/49051, WO00/46379, J. Biol. Chem., 271, 28271 (1996), J. Biol. Chem., 275, 3896 (2000), Mol. Microbiol., 39, 341 (2001)]. However, there is no report in which the gene is obtained from a microorganism belonging to the genus Pasteurella.

Furthermore, the α2,3-sialyltransferase derived from a microorganism belonging to the genus Campylobacter has also α2,8-sialyltransferase activity [WO00/46379, J. Biol. Chem., 275, 3896 (2000)]. However, it has not been known that there is a protein having α2,8-sialyltransferase activity in a microorganism belonging to the genus Pasteurella.

### DISCLOSURE OF THE INVENTION

Objects of the present invention are to provide a process for producing a protein having α2,3/α2,8-sialyltransferase activity using a transformant comprising a DNA encoding a protein having α2,3/α2,8-sialyltransferase activity and a process for producing a sialic acid-containing complex carbohydrate.

In order to solve the above problems, the present inventors have conducted intensive studies and obtained pm0188 gene product having homology with a known α2,6-sialyltransferase and pm0508 gene product and pm1174 gene product having homology with a known α2,3-sialyltransferase by searching the sequence information of Pasteurella multocida PM70 in which the sequence of the genomic DNA was determined. It was found that all gene products have α2,3/α2,8-sialyltransferase activity by studying the activity of each of the gene products in detail, the DNA was obtained, and thus the present invention has been completed.

Specifically, the present invention relates to the following (1) to (29):

(1) A process for producing a protein having α2,3/α2,8-sialyltransferase activity, which comprises: culturing a transformant capable of producing a protein having α2,3/α2,8-sialyltransferase activity derived from a microorganism belonging to the genus Pasteurella in a medium to produce and accumulate the protein having α2,3/α2,8-sialyltransferase activity in the culture, and recovering the protein from the culture.

(2) The process according to (1), wherein the microorganism belonging to the genus Pasteurella is Pasteurella multocida.

(3) The process according to (1), wherein the protein having α2,3/α2,8-sialyltransferase activity is a protein comprising the amino acid sequence represented by any one selected from SEQ ID NOs:1, 3 and 5.

(4) The process according to (1), wherein the protein having α2,3/α2,8-sialyltransferase activity is a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity.

(5) The process according to (1), wherein the protein having α2,3/α2,8-sialyltransferase activity is a protein which has an identity of at least 80% with the amino acid sequence represented by any one selected from SEQ ID NOs:1, 3 and 5, and has α2,3/α2,8-sialyltransferase activity.

(6) The process according to (1), wherein the transformant is a transformant which comprises a recombinant DNA comprising a DNA encoding a protein having α2,3/α2,8-sialyltransferase activity.

(7) The process according to (6), wherein the transformant is obtained by using a microorganism as a host cell.

(8) The process according to (7), wherein the microorganism is Escherichia coli.

(9) The process according to (6), wherein the DNA encoding the protein having α2,3/α2,8-sialyltransferase activity is a DNA described in one of the following (a) to (e):
(a) a DNA encoding a protein comprising the amino acid sequence represented by any one selected from SEQ ID NOs:1, 3 and 5;
(b) a DNA encoding a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity;
(c) a DNA encoding a protein which has an identity of at least 80% with the amino acid sequence represented by any one selected from SEQ ID NOs:1, 3 and 5, and has α2,3/α2,8-sialyltransferase activity;
(d) a DNA comprising the nucleotide sequence represented by any one selected from SEQ ID NOs:2, 4 and 6; and
(e) a DNA which hybridizes with a DNA consisting of a complementary sequence of the nucleotide sequence represented by any one selected from SEQ ID NOs:2, 4 and 6 under stringent conditions, and encodes a protein having α2,3/α2,8-sialyltransferase activity.

(10) A process for producing a sialic acid-containing complex carbohydrate, which comprises: allowing a culture of a transformant capable of producing a protein having α2,3/α2,8-sialyltransferase activity derived from a microorganism or a treated product of the culture as an enzyme source, cytidine-5′-monophosphosialic acid and an acceptor complex carbohydrate to be present in an aqueous medium to produce and accumulate the sialic acid-containing complex carbohydrate in the aqueous medium, and recovering the sialic acid-containing complex carbohydrate from the aqueous medium.

(11) The process according to (10), wherein the treated product of the culture is selected from the group consisting of a concentrated product of the culture, a dried product of the culture, cells obtained by centrifuging the culture, a dried product of the cells, a freeze-dried product of the cells, a surfactant-treated product of the cells, an ultrasonic-treated product of the cells, a mechanically disrupted product of the cells, a solvent-treated product of the cells, an enzyme-treated product of the cells, a protein fraction of the cells, an immobilized product of the cells and an enzyme preparation obtained by extracting from the cells.

(12) The process according to (10), wherein the acceptor complex carbohydrate is a complex carbohydrate comprising an oligosaccharide having galactose in its non-reducing terminal.

(13) The process according to (10), wherein the acceptor complex carbohydrate is a complex carbohydrate comprising an oligosaccharide having sialic acid in its non-reducing terminal.

(14) The process according to (12), wherein the oligosaccharide having galactose in its non-reducing terminal is an oligosaccharide selected from the group consisting of lactose, globotriose, N-acetyllactosamine, lacto-N-tetraose, lacto-N-neotetraose, Lewis^{a} and Lewis^{X}.

(15) The process according to (13), wherein the oligosaccharide having sialic acid in its non-reducing terminal is an oligosaccharide selected from the group consisting of NeuAcα2-3Galβ1-4Glc and NeuAcα2-3Galβ1-4GlcNAc.

(16) The process according to (10), wherein the microorganism is a microorganism belonging to the genus Pasteurella.

(17) The process according to (16), wherein the microorganism belonging to the genus Pasteurella is Pasteurella multocida.

(18) The process according to (10), wherein the protein having α2,3/α2,8-sialyltransferase activity is a protein comprising the amino acid sequence represented by any one selected from SEQ ID NOs:1, 3 and 5.

(19) The process according to (10), wherein the protein having α2,3/α2,8-sialyltransferase activity is a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity.

(20) The process according to (10), wherein the protein having α2,3/α2,8-sialyltransferase activity is a protein which has an identity of at least 80% with the amino acid sequence represented by any one selected from SEQ ID NOs:1, 3 and 5, and has α2,3/α2,8-sialyltransferase activity.

(21) The process according to (10), wherein the transformant is a transformant which comprises a recombinant DNA comprising a DNA encoding a protein having α2,3/α2,8-sialyltransferase activity.

(22) The process according to (21), wherein the transformant is obtained by using a microorganism as a host cell.

(23) The process according to (22), wherein the microorganism is Escherichia coli.

(24) The process according to (21), wherein the DNA encoding the protein having α2,3/α2,8-sialyltransferase activity is a DNA represented by one of the following (a) to (e):
(a) a DNA encoding a protein comprising the amino acid sequence represented by any one selected from SEQ ID NOs:1, 3 and 5;
(b) a DNA encoding a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity;
(c) a DNA encoding a protein which has an identity of at least 80% with the amino acid sequence represented by any one selected from SEQ ID NOs:1, 3 and 5, and has α2,3/α2,8-sialyltransferase activity;
(d) a DNA comprising the nucleotide sequence represented by any one selected from SEQ ID NOs:2, 4 and 6; and
(e) a DNA which hybridizes with a DNA consisting of a complementary sequence of the nucleotide sequence represented by any one selected from SEQ ID NOs:2, 4 and 6 under stringent conditions, and encodes a protein having α2,3/α2,8-sialyltransferase activity.

(25) A protein having α2,3/α2,8-sialyltransferase activity, which comprises the amino acid sequence represented by SEQ ID NO:1.

(26) A protein having α2,3/α2,8-sialyltransferase activity, which comprises the amino acid sequence represented by SEQ ID NO:3.

(27) A protein having α2,3/α2,8-sialyltransferase activity, which comprises the amino acid sequence represented by SEQ ID NO:5.

(28) A protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity.

(29) A protein which has an identity of at least 80% with the amino acid sequence represented by any one selected from SEQ ID NOs:1, 3 and 5, and has α2,3/α2,8-sialyltransferase activity.

The protein having α2,3/α2,8-sialyltransferase activity of the present invention includes the protein derived from a microorganism belonging to the genus Pasteurella, preferably the protein derived from Pasteurella multocida. The origin of the protein having α2,3/α2,8-sialyltransferase activity used in the process for producing a complex carbohydrate of the present invention is not particularly limited, so long as it is derived from a microorganism, and is preferably a protein derived from a microorganism belonging to the genus Pasteurella, and more preferably a protein derived from Pasteurella multocida. Specifically, the protein includes a protein comprising the amino acid sequence represented by any one selected from SEQ ID NOs:1, 3 and 5; a protein which consists of an amino acid sequence in which at least one amino acid is deleted, substituted or added in the amino acid sequence represented by any one, selected from SEQ ID NOs:1, 3 and 5, and has α2,3/α2,8-sialyltransferase activity; and a protein which has an identity of at least 80% with the amino acid sequence represented by any one selected from SEQ ID NOs:1, 3 and 5, and has α2,3/α2,8-sialyltransferase activity.

The protein which consists of an amino acid sequence in which at least one amino acid is deleted, substituted or added, and has α2,3/α2,8-sialyltransferase activity can be obtained, for example, by introducing mutation(s) to a DNA encoding a protein comprising the amino acid sequence represented by any one selected from SEQ ID NOs:1, 3 and 5 according to a method for introducing site-directed mutagenesis described in Molecular Cloning, A Laboratory Manual, Second Edition, Cold Spring Harbor Laboratory Press (1989) (hereinafter referred to as “Molecular Cloning, Second Edition”); Current Protocols in Molecular Biology, John Wiley & Sons (1987-1997) (hereinafter referred to as “Current Protocols in Molecular Biology”); Nucleic Acids. Research, 10, 6487 (1982); Proc. Natl. Acad. Sci. USA, 79, 6409 (1982); Gene, 34, 315 (1985); Nucleic Acids. Research, 13, 4431 (1985); Proc. Natl. Acad. Sci. USA, 82, 488 (1985) and the like.

The number of the amino acids which are deleted, substituted or added is not particularly limited; however, it is such a number that deletion, substitution or addition can be carried out by a known method such as method for introducing site-directed mutagenesis. The number is 1 to several tens, preferably 1 to 20, more preferably 1 to 10, and most preferably 1 to 5.

The deletion, substitution or addition of at least one amino acid residue in the amino acid sequence represented by any one selected from SEQ ID NOs:1, 3 and 5 means that one or at least two amino acids are deleted, substituted or added at any position in the same sequence. The deletion, substitution or addition can be carried out in the same amino acid sequence simultaneously. Also, the amino acid residue substituted, inserted or added can be natural or non-natural. The natural amino acid residue includes L-alanine, L-asparagine, L-aspartic acid, L-glutamine, L-glutamic acid, glycine, L-histidine, L-isoleucine, L-leucine, L-lysine, L-arginine, L-methionine, L-phenylalanine, L-proline, L-serine, L-threonine, L-tryptophan, L-tyrosine, L-valine, L-cysteine, and the like.

Herein, examples of amino acid residues which are substituted with each other are shown below. Amino acid residues in the same group can readily be substituted with each other.
Group A:
   leucine, isoleucine, norleucine, valine, norvaline, alanine, 2-aminobutanoic acid, methionine, O-methylserine, t-butylglycine, t-butylalanine, cyclohexylalanine;
Group B:
   aspartic acid, glutamic acid, isoaspartic acid, isoglutamic acid, 2-aminoadipic acid, 2-aminosuberic acid;
Group C:
   asparagine, glutamine;
Group D:
   lysine, arginine, ornithine, 2,4-diaminobutanoic acid, 2,3-diaminopropionic acid;
Group E:
   proline, 3-hydroxyproline, 4-hydroxyproline;
Group F:
   serine, threonine, homoserine;
Group G:
   phenylalanine, tyrosine.

Also, in order that the protein which consists of an amino acid sequence in which at least one amino acid is deleted, substituted or added has α2,3/α2,8-sialyltransferase activity, the protein has a homology of preferably at least 60% or more, more preferably 80% or more, and most preferably 95% or more, with the amino acid sequence represented by any one selected from SEQ ID NOs:1, 3 and 5.

The identity of an amino acid sequence or a nucleotide sequence can be determined by using the algorithm BLAST by Karlin and Altschul [Proc. Natl. Acad. Sci. USA, 90,5873 (1993)] or FASTA [Methods Enzymol., 183, 63 (1990)]. The programs called BLASTN and BLASTX have developed based on the above algorithm BLAST [J. Mol. Biol, 215, 403 (1990)]. In the case of analyzing a nucleotide sequence by BLASTN based on BLAST, for example, the parameter can be set to score 100, wordlength=12. Also, in the case of analyzing an amino acid sequence by BLASTX based on BLAST, for example, the parameter can be set to score=50, wordlength=3. When BLAST and Gapped BLAST programs are used, a default parameter of each program can be used. The specific analysis methods of using the above programs are known (http://www.ncbi.nlm.nih.gov.).

The transformant used in the production of the protein having α2,3/α2,8-sialyltransferase activity of the present invention is not particularly limited, so long as it is a transformant capable of producing a protein having α2,3/α2,8-sialyltransferase activity derived from a microorganism belonging to the genus Pasteurella. The transformant is preferably a transformant comprising a DNA encoding the protein derived from a microorganism belonging to the genus Pasteurella, and more preferably a transformant comprising a DNA encoding the protein derived from Pasteurella multocida. Examples include:
(1) a DNA encoding a protein comprising the amino acid sequence represented by SEQ ID NO:1;
(2) a DNA encoding a protein comprising the amino acid sequence represented by SEQ ID NO:3;
(3) a DNA encoding a protein comprising the amino acid sequence represented by SEQ ID NO:5;
(4) a DNA comprising the nucleotide sequence represented by SEQ ID NO:2;
(5) a DNA comprising the nucleotide sequence represented by SEQ ID NO:4;
(6) a DNA comprising the nucleotide sequence represented by SEQ ID NO:6;
(7) a DNA encoding a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity;
(8) a DNA encoding a protein which has the identity of at least 80% with the amino acid sequence represented by any one selected from SEQ ID NOs:1, 3 and 5, and has α2,3/α2,8-sialyltransferase activity; and
(9) a DNA which hybridizes with a DNA consisting of a complementary sequence of the nucleotide sequence represented by any one selected from SEQ ID NOs:2, 4 and 6 under stringent conditions, and encodes a protein having α2,3/α2,8-sialyltransferase activity.

The DNA which is hybridizable under stringent conditions is a DNA obtained by colony hybridization, plaque hybridization, Southern hybridization or the like using, as a probe, a part or a full length of a DNA consisting of a complementary sequence of the nucleotide sequence represented by any one selected from SEQ ID NOs:2, 4 and 6. Specifically, the DNA includes a DNA which can be identified by carrying out hybridization at 65° C. in the presence of 0.7-1.0 mol/l NaCl using a filter on which a DNA prepared from colonies or plaques is immobilized, and then washing the filter with 0.1× to 2×SSC solution (the composition of 1×SSC solution contains 150 mmol/l sodium chloride and 15 mmol/l sodium citrate) at 65° C. The hybridization can be carried out in accordance with a known method described in, for example, Molecular Cloning, Second Edition; Current Protocols in Molecular Biology; DNA Cloning 1: Core Techniques, A Practical Approach, Second Edition, Oxford University (1995) or the like.

Specifically, the DNA which is hybridizable includes a DNA having a homology of at least 60% or more, preferably 80% or more, and more preferably 95% or more, with the nucleotide sequence represented by any one selected from SEQ ID NOs:2, 4 and 6 when calculated based on the above parameters using above BLAST, FASTA or the like.

[1] Preparation of a DNA Used in the Process for Producing the Protein Having α2,3/α2,8-Sialyltransferase Activity of the Present Invention

(1) Selection of a DNA Encoding the Protein Having α2,3/α2,8-Sialyltransferase Activity Using Database

The full nucleotide sequence of the genomic DNA in Pasteurella multocida PM70 was determined [Proc. Natl. Acad. Sci., USA, 98, 3460 (2001)], and the DNA encoding the protein having α2,3/α2,8-sialyltransferase activity can be selected by carrying out gene search, homology search and the like by using a known sialyltransferase gene based on the genomic DNA sequence database [http://www.cbc.umn.edu/ResearchProjects/Pm/pmhome.html, http://www.ncbi.nlm.nih.gov/BLAST/].

(2) Preparation of a DNA Encoding the Protein Having α2,3/α2,8-Sialyltransferase Activity

The DNA encoding the protein having α2,3/α2,8-sialyltransferase activity can be prepared from a microorganism belonging to the genus Pasteurella. The microorganism belonging to the genus Pasteurella includes Pasteurella multocida, and specifically Pasteurella multocida PM70 (available from Minnesota University) and the like.

The microorganism belonging to Pasteurella multocida can be cultured by a known method [for example, FEMS Microbiol. Lett., 166, 289 (1998)].

After the culturing, a chromosomal DNA of the microorganism can be isolated and purified by a known method (for example, method described in Current Protocols in Molecular Biology).

A DNA fragment containing the DNA used in the production process for production of the present invention can be obtained by preparing a primer based on the nucleotide sequence of the genomic DNA selected in the item (1) and then carrying out PCR [PCR Protocols, Academic Press (1990)] using the genomic DNA as a template.

Furthermore, the DNA of interest can be obtained according to a hybridization method by using the synthetic DNA designed based on the nucleotide sequence of the genomic DNA as a probe.

The nucleotide sequence of the DNA can be determined by inserting the obtained DNA as it is or after digestion with an appropriate restriction enzyme, into a vector according to the usual method, and carrying out analysis by the generally used nucleotide sequence analysis method such as the dideoxy method [Proc. Natl. Acad. Sci. USA, 74, 5463 (1977)] or a method comprising the use of an apparatus for nucleotide sequence analysis such as 373A-DNA Sequencer (manufactured by Perkin-Elmer).

Based on the nucleotide sequence thus determined, the DNA of interest can also be prepared by chemical synthesis using, for example, DNA Synthesizer 8905 manufactured by Perceptive Biosystems or the like.

The thus obtained DNA includes a DNA comprising the sequence represented by SEQ ID NO:2, 4 or 6.

The vector into which the DNA of the present invention is ligated includes pBluescript II KS(+) (manufactured by Stratagene), pDIRECT [Nucleic Acids Res., 18, 6069 (1990)], pCR-Script Amp SK(+) (manufactured by Stratagene), pT7Blue manufactured by Novagen), pCR II (manufactured by Invitrogen), pCR-TRAP (manufactured by Genehunter) and the like.

Recombinant DNAs pPM0188SK, pPM0508SK and pPM1174SK which comprise the DNA comprising the nucleotide sequence represented by SEQ ID NO:2, 4 or 6, respectively, have been deposited on Sep. 13, 2001, as FERM BP-7730, FERM BP-7731 and FERM BP-7733, respectively, in International Patent Organism Depositary, National Institute of Advanced Industrial Science and Technology (Central 6, 1-1, Higashi 1-Chome Tsukuba, Ibaraki, 305-8566, Japan).

The microorganism containing the recombinant DNA which comprises the DNA comprising the sequence represented by SEQ ID NO:2, 4 or 6 includes Escherichia coli and the like.

Escherichia coli includes Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No.49, Escherichia coli W3110, Escherichia coli NY49, Escherichia coli MP347, Escherichia coli NM522, Escherichia coli ME8415 and the like.

Any method can be used in the introduction method of the recombinant DNA, so long as it is a method for introducing DNA into the host cell. Examples include the method using a calcium ion [Proc. Natl. Acad. Sci. USA, 69, 2110 (1972)], the protoplast method (Japanese Published Unexamined Patent Application No. 248394/88), electroporation [Nucleic Acid Res., 16, 6127 (1988)] and the like.

The transformant capable of producing α2,3/α2,8-sialyltransferase obtained by introducing the above recombinant DNA includes a transformant containing the above recombinant DNA, a transformant into which a DNA encoding α2,3/α2,8-sialyltransferase has been integrated into a chromosomal DNA of the host cell by homologous recombination, and the like.

The transformant containing the recombinant DNA includes Escherichia coli NM522/pPT76, Esherichia coli NM522/pPT79 and Esherichia coli NM522/pPT78 which are Esherichia coli containing the recombinant DNA which comprises the DNA comprising the nucleotide sequence represented by SEQ ID NO:2, 4 or 6, respectively.

[2] Preparation of the Protein Having α2,3/α2,8-Sialyltransferase Activity

The protein having α2,3/α2,8-sialyltransferase activity can be produced by expressing the DNA obtained by the method of the above item [1] in a host cell, for example, as shown below, by using a method described in Molecular Cloning, Second Edition, Current Protocols in Molecular Biology or the like.

Based on the above DNA, a DNA fragment of an appropriate length containing a portion which encodes the protein can be prepared, if necessary. In addition, productivity of the protein can be improved by substituting a nucleotide in the nucleotide sequence of the protein-coding region so that it has the most suitable codons for the expression in the host.

A recombinant DNA is prepared by inserting the DNA into a downstream of the promoter of an appropriate expression vector.

A transformant capable of producing the protein used for the process of the present invention can be obtained by introducing the recombinant DNA into a host cell suitable for the expression vector.

Any bacteria, yeasts, animal cells, insect cells, plant cells and the like can be used as the host cell, so long as it can express the gene of interest.

The expression vectors include those which can replicate autonomously in the above host cell or those which can be integrated into a chromosome and have a promoter at such a position that the DNA encoding the protein having α2,3/α2,8-sialyltransferase activity can be transcribed.

When a procaryote such as bacterium is used as the host cell, it is preferred that the recombinant DNA which comprises the DNA encoding the protein used in the process for production of present invention can replicate autonomously in the procaryote, and that the recombinant vector contains a promoter, a ribosome binding sequence, the DNA of the present invention and a transcription termination sequence. The vector may further comprise a gene regulating the promoter.

The expression vector includes pHelix1 (manufactured by Roche Diagnostics), pKK233-2 (manufactured by Amersham Pharmacia Biotech), pSE280 (manufactured by Invitrogen), pGEMEX-1 (manufactured by Promega), pQE-8 (manufactured by QIAGEN), pET-3 (manufactured by Novagen), pKYP10 (Japanese Published Unexamined Patent Application No. 110600/83), pKYP200 [Agric. Biol. Chem., 48, 669 (1984)], pLSA1 [Agric. Biol. Chem., 53, 277 (1989)], pGEL1 [Proc. Natl. Acad. Sci. USA, 82, 4306 (1985)], pBluescript II SK(+), pBluescript II KS(−) (manufactured by Stratagene), pTrS30 [prepared from Escherichia coli JM109/pTrS30 (FERM BP-5407)], pTrS32 [prepared from Escherichia coli JM109/pTrS32 (FERM BP-5408)], pPAC31 (WO98/12343), pUC19 [Gene, 33, 103 (1985)], pSTV28 (manufactured by Takara Shuzo), pUC118 (manufactured by Takara Shuzo), pPA1 (Japanese Published Unexamined Patent Application No. 233798/88) and the like.

Any promoter can be used, so long as it can function in the host cell. Examples include promoters derived from Escherichia coli, phage and the like, such as trp promoter (Pₜᵣₚ), lac promoter (P_{lac}), P_{L} promoter, P_{R} promoter and P_{SE} promoter, SPO1 promoter, SPO2 promoter, penP promoter and the like. Also, artificially designed and modified promoters, such as a promoter in which two Pₜᵣₚ are linked in tandem, tac promoter, lacT7 promoter and letI promoter, can be used.

It is preferred to use a plasmid in which the space between Shine-Dalgarno sequence, which is the ribosome binding sequence, and the initiation codon is adjusted to an appropriate distance (for example, 6 to 18 nucleotides).

The transcription termination sequence to express the protein having α2,3/α2,8-sialyltransferase activity is not essential for the recombinant DNA. However, it is preferred to lie a transcription terminating sequence immediately downstream of the structural gene.

The procaryotes include microorganisms belonging to the genera Escherichia, Serratia, Bacillus, Brevibacterium, Corynebacterium, Microbacterium, Pseudomonas and the like. Examples include Escherichia coli XL1-Blue, Escherichia coli XL2-Blue, Escherichia coli DH1, Escherichia coli NM522, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Serratia ficaria, Serratia fonticola, Serratia liquefaciens, Serratia marcescens, Bacillus subtilis, Bacillus amyloliquefaciens, Brevibacterium immariophilum ATCC 14068, Brevibacterium saccharolyticum ATCC 14066, Corynebacterium ammoniagenes, Corynebacterium glutamicum ATCC 13032, Corynebacterium glutamicum ATCC 14067, Corynebacterium glutamicum ATCC 13869, Corynebacterium acetoacidophilum ATCC 13870, Microbacterium ammoniaphilum ATCC 15354, Pseudomonas sp. D-0110 and the like.

Introduction of the recombinant DNA can be carried out by any methods for introducing DNA into the above-described host cells, such as the method using a calcium ion [Proc. Natl. Acad. Sci. USA, 69, 2110 (1972)], the protoplast method (Japanese Published Unexamined Patent Application No. 248394/88) and electroporation [Nucleic Acids Res., 16, 6127 (1988)].

When a yeast cell is used as the host cell, the expression vector includes YEp13 (ATCC 37115), YEp24 (ATCC 37051), YCp50 (ATCC 37419), pHS19, pHS15 and the like.

Any promoter can be used so long as it can function in yeast. Examples include PHO5 promoter, PGK promoter, GAP promoter, ADH promoter, gal 1 promoter, gal 10 promoter, a heat shock polypeptide promoter, MFα1 promoter, CUP 1 promoter and the like.

The host cell includes yeast strain belonging to the genera Saccharomyces, Schizosaccharomyces, Kluyveromyces, Trichosporon, Schwanniomyces, Pichia, Candida and the like. Examples include Saccharomyces cerevisiae, Schizosaccharomyces pombe, Kluyveromyces lactis, Trichosporon pullulans, Schwanniomyces alluvius, Pichia pastoris, Candida utilis and the like.

Introduction of the recombinant DNA can be carried out by any of the methods for introducing DNA into yeast, such as electroporation [Methods. Enzymol., 194, 182 (1990)], the spheroplast method [Proc. Natl. Acad. Sci. USA, 81, 4889 (1984)] and the lithium acetate method [J. Bacteriol., 153, 163 (1983)].

When an animal cell is used as the host, the expression vector includes pcDNAI (available from Funakoshi) and pcDM8 (available from Funakoshi), pAGE107 (Japanese Published Unexamined Patent Application No. 22979/91), pAS3-3 (Japanese Published Unexamined Patent Application No. 227075/90), pcDNAI/Amp (manufactured by Invitrogen), pREP4 (manufactured by Invitrogen), pAGE103 [J. Biochem., 101, 1307 (1987)], pAGE210, pAMo, pAMoA and the like.

Any promoter can be used, so long as it can function in an animal cell. Examples include a promoter of IE (immediate early) gene of cytomegalovirus (CMV), SV40 early promoter, a metallothionein promoter, a promoter of retrovirus, a heat shock promoter, SRα promoter and the like. Also, the enhancer of the IE gene of human CMV can be used together with the promoter.

The host cell includes mouse myeloma cell, rat myeloma cell, mouse hybridoma cell, human Namalwa cell, Namalwa KIM-1 cell, human fetal kidney cell, human leukemia cell, African grivet kidney cell, Chinese hamster ovary (CHO) cell, HST5637 (Japanese Published Unexamined Patent Application No. 299/88) and the like.

The mouse myeloma cell includes SP2/0, NS0 and the like. The rat myeloma cell includes YB2/0 and the like. The human fetal kidney cell includes HEK293 (ATCC: CRL-1573) and the like. The human leukemia cell includes BALL-1 and the like. The African grivet kidney cell includes COS-1, COS-7 and the like.

Introduction of the recombinant DNA into animal cells can be carried out by any of methods for introducing DNA into animal cells, such as electroporation [Cytotechnology, 3, 133 (1990)], the calcium phosphate method (Japanese Published Unexamined Patent Application No. 227075/90), the lipofection method [Proc. Natl. Acad. Sci. USA, 84, 7413 (1987)], and the method described in Virology, 52, 456 (1973).

When an insect cell is used as the host, the protein can be produced by a known method described in, for example, Baculovirus Expression Vectors, A Laboratory Manual, W. H. Freeman and Company, New York (1992), Molecular Biology, A Laboratory Manual, Current Protocols in Molecular Biology, Bio/Technology, 6, 47 (1988) or the like.

Specifically, a recombinant gene transfer vector and baculovirus are co-transfected into an insect cell to obtain a recombinant virus in a supernatant of the culture of its insect cell, and then an insect cell is infected with the resulting recombinant virus to produce the protein.

The transfer vector used in the method includes pVL1392, pVL1393 and pBlueBacIII (all manufactured by Invitrogen), and the like.

The baculovirus includes Autographa californica nuclear polyhedrosis virus which infects insects of the family Barathra and the like.

The insect cell includes Spodoptera frugiperda ovary cell, Trichoplusia ni ovary cell, silkworm ovary-derived culturing cell and the like.

Spodoptera frugiperda ovary cell includes Sf9 and Sf21 (Baculovirus Expression Vectors, A Laboratory Manual) and the like. Trichoplusia ni ovary cell includes High 5 and BTI-TN-5B1-4 (manufactured by Invitrogen) and the like. The cell line derived from silkworm ovary cell includes Bombyx mori N4 and the like.

The method for co-transfecting the above transfer vector and the above baculovirus for the preparation of the recombinant virus includes the calcium phosphate method (Japanese Published Unexamined Patent Application No. 227075/90), the lipofection method [Proc. Natl. Acad. Sci. USA, 84, 7413 (1987)] and the like.

When a plant cell is used as the host cell, the expression vector includes Ti plasmid, a tobacco mosaic virus vector, and the like.

As the promoter, any promoter can be used, so long as it can function in a plant cell. Examples include 35S promoter of cauliflower mosaic virus (CaMV), rice actin 1 promoter and the like.

The host cell includes a plant cell and the like, such as tobacco, potato, tomato, carrot, soybean, rape, alfalfa, rice, wheat and barley.

Introduction of the recombinant vector is carried out by any of methods for introducing DNA into a plant cell, such as Agrobacterium method (Japanese Published Unexamined Patent Application No. 140885/84, Japanese Published Unexamined Patent Application No. 70080/85, WO 94/00977), electroporation (Japanese Published Unexamined Patent Application No. 251887/85) and the method using a particle gun (Japanese Patent Nos. 2606856 and 2517813).

The protein having α2,3/α2,8-sialyltransferase activity can be produced by culturing the transformant thus obtained in a medium to produce and accumulate the protein having α2,3/α2,8-sialyltransferase activity in the culture, and recovering it from the culture.

Culturing the transformant used in the process for producing the protein having α2,3/α2,8-sialyltransferase activity in a medium is carried out according to the conventional method as used in culturing of the host.

As a medium for culturing the transformant obtained by using, as the host, prokaryote such as Escherichia coli, or eukaryote such as yeast, either a natural medium or a synthetic medium may be used, so long as it contains a carbon source, a nitrogen source, an inorganic salt and the like which can be assimilated by the organism and the transformant can be cultured efficiently.

Any carbon source can be used, so long as the organism can assimilate, and it includes carbohydrates, such as glucose, fructose, sucrose, molasses containing them, starch and starch hydrolysate; organic acids, such as acetic acid and propionic acid; alcohols, such as ethanol and propanol; and the like.

The nitrogen source includes ammonia, various ammonium salts of inorganic acids or organic acids, such as ammonium chloride, ammonium sulfate, ammonium acetate and ammonium phosphate; other nitrogen-containing compounds; peptone; meat extract; yeast extract; corn steep liquor; casein hydrolysate; soybean meal and soybean meal hydrolysate; various fermented cells and digested matter thereof; and the like.

The inorganic salt includes potassium dihydrogen phosphate, dipotassium hydrogen phosphate, magnesium phosphate, magnesium sulfate, sodium chloride, ferrous sulfate, manganese sulfate, copper sulfate, calcium carbonate and the like.

Culturing is usually carried out under aerobic conditions by shaking culture, submerged spinner culture under aeration or the like. The culturing temperature is preferably from 15 to 40° C., and the culturing time is generally from 5 hours to 7 days. The pH of the medium is preferably maintained at 3.0 to 9.0 during the culturing. The pH can be adjusted using inorganic or organic acid, an alkali solution, urea, calcium carbonate, ammonia or the like.

Also, antibiotics, such as ampicillin and tetracycline, can be added to the medium during culturing, if necessary.

When a microorganism transformed with an expression vector containing an inducible promoter is cultured, an inducer can be added to the medium, if necessary. For example, isopropyl-β-D-thiogalactopyranoside or the like can be added to the medium when a microorganism transformed with an expression vector containing lac promoter is cultured; or indoleacrylic acid or the like can be added thereto when a microorganism transformed with an expression vector containing trp promoter is cultured.

The medium for culturing a transformant obtained using an animal cell as the host includes generally used RPMI 1640 medium [The Journal of the American Medical Association, 199, 519 (1967)], Eagle's MEM medium [Science, 122, 501 (1952)], DMEM medium [Virology, 8, 396 (1959)], and 199 Medium [Proceeding of the Society for the Biological Medicine, 73, 1 (1950)], as well as media to which fetal calf serum or the like has been added to the above media and the like.

Culturing is generally carried out at pH 6 to 8 and at 25 to 40° C. for 1 to 7 days in the presence of 5% CO₂ or the like.

Furthermore, if necessary, antibiotics such as kanamycin, penicillin and streptomycin, can be added to the medium during the culturing.

The medium for culturing a transformant obtained using an insect cell as the host includes generally used TNM-FH medium (manufactured by Pharmingen), Sf-900 II SFM (manufactured by Life Technologies), ExCell 400 and ExCell 405 (both manufactured by JRH Biosciences), Grace's Insect Medium [Nature, 195, 788 (1962)] and the like.

Culturing is generally carried out at pH 6 to 7 and at 25 to 30° C. for 1 to 5 days or the like.

Furthermore, if necessary, antibiotics such as gentamicin can be added to the medium during the culturing.

A transformant obtained by using a plant cell as the host cell can be used as the cell or after differentiating to a plant cell or organ. The medium used in the culturing of the transformant includes Murashige and Skoog (MS) medium, White medium, media to which a plant hormone, such as auxin or cytokinine, has been added, and the like.

Culturing is carried out generally at a pH 5 to 9 and at 20 to 40° C. for 3 to 60 days.

Also, antibiotics, such as kanamycin and hygromycin, can be added to the medium during the culturing, if necessary.

As described above, the protein having α2,3/α2,8-sialyltransferase activity can be produced by culturing a transformant derived from a microorganism, animal cell, insect cell or plant cell containing the recombinant DNA which comprises the DNA encoding the protein according to the general culturing method to produce and accumulate the protein, and recovering the protein from the culture.

In the production of the protein having α2,3/α2,8-sialyltransferase activity, the protein can be produced so as to have a structure as it is or can be produced as a secretory protein having a signal sequence or a fusion protein according to the method described in Molecular Cloning, Second Edition and the like.

The protein to be fused includes β-galactosidase, protein A, IgG binding region of protein A, chloramphenicol acetylransferase, poly(Arg), poly(Glu), protein G maltose binding protein, glutathione S-transferase, polyhistidine chain (His-tag), S peptide, DNA binding protein domain, Tac antigen, thioredoxin, green fluorescent protein, FLAG peptide, epitope of any antibody, and the like [Akio Yamakawa, Experimental Medicine (Jikken Igaku), 13, 469-474 (1995)].

Furthermore, the process for producing the protein having α2,3/α2,8-sialyltransferase activity includes a process for production in which the protein is produced on an outer membrane of the host cell, and the process for production can be selected by changing the host cell used or the structure of the protein produced.

When the protein having α2,3/α2,8-sialyltransferase activity is produced in a host cell or on an outer membrane of the host cell, the produced protein can be actively secreted extracellularly according to, for example, the method of Paulson et al. [J. Biol. Chem., 264, 17619 (1989)], the method of Lowe et al. [Proc. Natl. Acad. Sci. USA, 86, 8227 (1989); Genes Develop., 4, 1288 (1990)], or the methods described in Japanese Published Unexamined Patent Application No. 336963/93, WO94/23021, and the like.

Specifically, the protein having α2,3/α2,8-sialyltransferase activity can be actively secreted extracellularly by producing it in the form that a signal peptide has been added to the side of N-terminal of a protein containing an active site of the protein having α2,3/α2,8-sialyltransferase activity according to the recombinant DNA technique.

Furthermore, the protein production can be increased utilizing a gene amplification system using a dihydrofolate reductase gene or the like according to the method described in Japanese Published Unexamined Patent Application No. 227075/90.

Moreover, the protein having α2,3/α2,8-sialyltransferase activity can be produced by rediferentiating a gene-introduced animal or plant cell to develop a gene-introduced transgenic animal individual (transgenic nonhuman animal) or plant individual (transgenic plant), and using the individual.

When the transformant is the animal individual or plant individual, the protein can be produced by breeding or cultivating it to produce and accumulate the protein, and recovering the protein from the animal individual or plant individual.

The process for producing the protein having α2,3/α2,8-sialyltransferase activity using the animal individual includes a method for producing the protein of the present invention in a nonhuman animal developed by introducing a gene according to a known method [Am. J. Clin. Nutr, 63, 639S (1996), Am. J. Clin. Nutr, 63, 627S (1996), Bio/Technology, 9, 830 (1991)].

In the animal individual, the protein can be produced by breeding a transgenic nonhuman animal to which the DNA encoding the protein having α2,3/α2,8-sialyltransferase has been introduced to produce and accumulate the protein in the animal, and recovering the protein from the animal. The protein produced in the animal is accumulated in milk (Japanese Published Unexamined Patent Application No. 309192/88), egg, and the like. Any promoter can be used, so long as it can function in the animal. Suitable examples include an α-casein promoter, a β-casein promoter, a β-lactoglobulin promoter, a whey acidic protein promoter, and the like, which are specific for mammary glandular cells.

The process for producing the protein having α2,3/α2,8-sialyltransferase activity using the plant individual includes a process for producing the protein by cultivating a transgenic plant to which the DNA encoding the protein of the present invention is introduced by a known method [Tissue Culture (Soshiki Baiyo), 20 (1994), Tissue Culture (Soshiki Baiyo), 21 (1995), Trends Biotechnol., 15, 45 (1997)] to produce and accumulate the protein in the plant, and recovering the protein from the plant.

The protein having α2,3/α2,8-sialyltransferase activity produced by the production process of the protein of the present invention can be isolated and purified by using the general method for isolating and purifying an enzyme.

For example, when the protein having α2,3/α2,8-sialyltransferase activity is produced as a soluble product in the host cells, the cells are collected by centrifugation after culturing, suspended in an aqueous buffer, and disrupted using an ultrasonicator, a French press, a Manton Gaulin homogenizer, a Dynomill, or the like to obtain a cell-free extract.

From the supernatant obtained by centrifuging the cell-free extract, a purified product can be obtained by the general method used for isolating and purifying an enzyme, for example, solvent extraction, salting-out using ammonium sulfate or the like, desalting, precipitation using an organic solvent, anion exchange chromatography using a resin such as diethylaminoethyl (DEAE)-Sepharose or DIAION HPA-75 (manufactured by Mitsubishi Chemical), cation exchange chromatography using a resin such as S-Sepharose FF (manufactured by Pharmacia), hydrophobic chromatography using a resin such as butyl sepharose or phenyl sepharose, gel filtration using a molecular sieve, affinity chromatography, chromatofocusing, or electrophoresis such as isoelectronic focusing, alone or in combination thereof.

When the protein is produced as an inclusion body in the host cells, the cells are collected in the same manner, disrupted and centrifuged to recover the protein as the precipitate fraction, and then the inclusion body of the protein is solubilized with a protein-denaturing agent.

The solubilized solution is diluted or dialyzed in a solution free from a protein denaturing agent or a solution having a diluted concentration of a protein denaturing agent in such a degree that the protein is not denatured to thereby constitute the normal tertiary structure of the protein, and then a purified product of the protein can be obtained by a purification/isolation method similar to the above.

When the protein having α2,3/α2,8-sialyltransferase activity or derivatives such as its glycosylated-derivatives are secreted out of cells, the protein or its derivatives such as the glycosylated-derivatives can be collected in the culture supernatant.

Specifically, the culture medium is treated in a manner similar to the above, such as centrifugation to obtain a solubilized fraction, from which a purified product can be obtained using a purification/isolation method similar to the above.

The protein obtained by the above method includes a protein comprising the amino acid sequence represented by SEQ ID NO:1, 3 or 5.

Furthermore, the protein having α2,3/α2,8-sialyltransferase activity is produced as a fusion protein with other protein, and can be purified using affinity chromatography using a substance having affinity to the fusion protein. For example, the fusion protein having α2,3/α2,8-sialyltransferase activity is produced as a fusion protein with protein A according to the method of Lowe et al. [Proc. Natl. Acad. Sci. USA, 86, 8227 (1989); Genes Develop., 4, 1288 (1990)], or the method described in Japanese Published Unexamined Patent Application No. 336963/93 or WO94/23021, and the fusion protein can be purified by affinity chromatography using immunoglubulin G.

Moreover, the protein having α2,3/α2,8-sialyltransferase activity is produced as a fusion protein with Flag peptide, and the fusion protein can be purified by affinity chromatography using an anti-Flag antibody [Proc. Natl. Acad. Sci., USA, 86, 8227 (1989), Genes Develop., 4, 1288 (1990)]. In addition, purification can be carried out by affinity chromatography using the antibody against the polypeptide per se.

Based on the amino acid sequence information of the protein thus obtained, the protein having α2,3/α2,8-sialyltransferase can be produced by a chemical synthesis method, such as Fmoc (fluorenylmethyloxycarbonyl) method or tBoc (t-butyloxycarbonyl) method. It can also be chemically synthesized using a peptide synthesizer manufactured by Advanced ChemTech, Perkin-Elmer, Pharmacia, Protein Technology Instrument, Synthecell-Vega, PerSeptive, Shimadzu Corporation, or the like.

(3) Preparation of Sialic Acid-Containing Complex Carbohydrate

The process for producing a sialic acid-containing complex carbohydrate of the present invention is a process for production, which comprises: allowing a culture of a transformant capable of producing a protein having α2,3/α2,8-sialyltransferase activity derived from a microorganism or a treated product of the culture as an enzyme source, cytidine-5′-monophosphosialic acid and an acceptor complex carbohydrate to be present in an aqueous medium to produce and accumulate the sialic acid-containing complex carbohydrate in the aqueous medium, and recovering the sialic acid-containing complex carbohydrate from the aqueous medium.

The transformant used in the production process of the sialic acid-containing complex carbohydrate of the present invention is not particularly limited, so long as it is a transformant capable of producing a protein having α2,3/α2,8-sialyltransferase derived from a microorganism. The transformant is preferably a transformant comprising a DNA encoding a protein having α2,3/α2,8-sialyltransferase activity derived from a microorganism belonging to the genus Pasteurella, and more preferably a transformant comprising a DNA encoding the protein derived from Pasteurella multocida.

The DNA encoding the protein derived from Pasteurella multocida includes:
(1) a DNA encoding a protein comprising the amino acid sequence represented by SEQ ID NO:1;
(2) a DNA encoding a protein comprising the amino acid sequence represented by SEQ ID NO:3;
(3) a DNA encoding a protein comprising the amino acid sequence represented by SEQ ID NO:5;
(4) a DNA comprising the nucleotide sequence represented by SEQ ID NO:2;
(5) a DNA comprising the nucleotide sequence represented by SEQ ID NO:4;
(6) a DNA comprising the nucleotide sequence represented by SEQ ID NO:6;
(7) a DNA encoding a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity; and
(8) a DNA which hybridizes with a DNA consisting of a complementary sequence of the nucleotide sequence represented by any one selected from SEQ ID NOs:2, 4 and 6 under stringent conditions, and encodes a protein having α2,3/α2,8-sialyltransferase activity.

The above-described DNA can be obtained by the method of the above item [1], and a transformant comprising the DNA and a culture of the transformant can be obtained by the method of the above item [2].

The treated product of the culture includes a concentrated product of the culture, a dried product of the culture, cells obtained by centrifuging the culture, a dried product of the cells, a freeze-dried product of the cells, a surfactant-treated product of the cells, an ultrasonic-treated product of the cells, a mechanically disrupted product of the cells, a solvent-treated product of the cells, an enzyme-treated product of the cells, a protein fraction of the cells, an immobilized product of the cells, an enzyme preparation obtained by extracting from the cell, and the like.

The enzyme source used in the production of the sialic acid-containing complex carbohydrate is used in a concentration of 1 mU/l to 1,000 U/l, preferably 10 mU/l to 500 U/l, when the activity capable of forming 1 μmol of sialic acid-containing complex carbohydrate at 37° C. in 1 minute is defined as 1 unit (U).

The aqueous medium used in the production of the sialic acid-containing complex carbohydrate includes water; a buffer such as a phosphate buffer, a carbonate buffer, an acetate buffer, a borate buffer, a citrate buffer and a tris buffer; alcohol, such as methanol and ethanol; ester such as ethyl acetate; ketone such as acetone; amide such as acetamide; and the like. Also, the culture of the microorganisms used as the enzyme source can be used as an aqueous medium.

The acceptor complex carbohydrate used in the production of the sialic acid-containing complex carbohydrate includes a complex carbohydrate comprising an oligosaccharide having galactose in its non-reducing terminal and a complex carbohydrate comprising an oligosaccharide having sialic acid in its non-reducing terminal, and is preferably a complex carbohydrate comprising an oligosaccharide having a structure selected from the group consisting of lactose, globotriose, N-acetyllactosamine, lacto-N-tetraose, lacto-N-neotetraose, Lewis^{a} and Lewis^{X} in its non-reducing terminal or a complex carbohydrate comprising an oligosaccharide having a structure selected from the group consisting of NeuAcα2-3Galβ1-4Glc and NeuAcα2-3Galβ1-4GlcNAc in its non-reducing terminal.

In the production of the sialic acid-containing complex carbohydrate, a surfactant or an organic solvent may be added, if necessary. Any surfactant capable of accelerating the formation of a sialic acid-containing complex carbohydrate can be used as the surfactant. Examples include nonionic surfactants such as polyoxyethylene octadecylamine (e.g., Nymeen S-215, manufactured by Nippon Oil & Fats); cationic surfactants, such as cetyltrimethylammonium bromide and alkyldimethyl benzylammoniumchloride (e.g., Cation F2-40E, manufactured by Nippon Oil & Fats); anionic surfactants such as lauroyl sarcosinate; tertiary amines such as alkyldimethylamine (e.g., Tertiary Amine FB, manufactured by Nippon Oil & Fats); and the like, which are used alone or as a mixture of two or more. The surfactant is used generally in a concentration of 0.1 to 50 g/l. The organic solvent includes xylene, toluene, fatty acid alcohol, acetone, ethyl acetate, and the like, which are used in a concentration of generally 0.1 to 50 ml/l.

The production reaction for the sialic acid-containing complex carbohydrate is carried out in an aqueous medium having a pH 5 to 10, preferably pH 6 to 8, at 20 to 50° C. for 1 to 96 hours. In the production reaction, inorganic salts, such as MnCl₂ and MgCl₂, can be added, if necessary.

The amount of the sialic acid-containing complex carbohydrate produced in the aqueous medium can be determined, for example, using a carbohydrate analysis system manufactured by Dionex [Anal. Biochem., 189:, 151 (1990)] or the like.

The sialic acid-containing complex carbohydrate produced in the aqueous medium can be recovered by the ordinary method using activated carbon, an ion exchange resin or the like.

Examples of the present invention are shown below; however, the present invention is not limited to these Examples.

### BRIEF DESCRIPTION OF THE DRAWING

FIG. 1 shows construction steps of an α2,3/α2,8-sialyltransferase gene expression plsmid pPT76.

FIG. 2 shows construction steps of an α2,3/α2,8-sialyltransferase gene expression plsmid pPT79.

FIG. 3 shows construction steps of an α2,3/α2,8-sialyltransferase gene expression plsmid pPT78.

Also, in the drawings, Amp represents an ampicillin-resistant gene; P_{L} represents P_{L} promoter; cI857 represents a temperature-sensitive repressor gene; pm0188 represents a gene encoding an α2,3/α2,8-sialyltransferase; pm0508 represents a gene encoding an α2,3/α2,8-sialyltransferase; and pm1174 represents a gene encoding an α2,3/α2,8-sialyltransferase.

### BEST MODE FOR CARRYING OUT THE INVENTION

### EXAMPLE 1

### Construction of a Strain Expressing a Gene Encoding an α2,3/α2,8-Sialyltransferase Derived From Pasteurella Multocida

(1) Construction of Escherichia Coli NM522/pPT76

A chromosomal DNA of Pasteurella multocida PM70 was obtained from Minnesota University.

Using DNA fragments having the nucleotide sequences represented by SEQ ID NOs:7 and 8 which had been synthesized by using a DNA synthesizer Model 8905 manufactured by Perceptive Biosystems, a DNA fragment containing pm0188 considered to be a gene encoding an α2,6-sialyltransferase in the full nucleotide sequence of the genomic DNA in Pasteurella multocida PM70 was amplified by the following method.

PCR was carried out by using the above synthetic DNA fragments as a primer set and using the chromosomal DNA of Pasteurella multocida PM70 as the template. The PCR was carried out by using 40 μl of a reaction solution containing 0.1 μg of the chromosomal DNA, 0.5 μmol/l of each of the primers, 2.5 units of Pfu DNA polymerase (manufactured by Stratagene), 4 μl of 10×buffer for Pfu DNA polymerase and 200 μmol/l of each deoxy NTP, and repeating 30 times of a step consisting of 1 minute at 94° C., 2 minutes at 42° C. and 3 minutes at 72° C.

A 1/10 volume of the reaction solution was subjected to agarose gel electrophoresis to confirm that the fragment of interest was amplified, and then the remaining reaction solution was mixed with the same volume of TE [10 mmol/l Tris-HCl and 1 mmol/l EDTA (pH 8.0)] saturated phenol/chloroform (1 vol/1 vol).

After centrifugation of the mixed solution, the thus obtained upper layer was mixed with 2 volumes of cold ethanol and allowed to stand at −80° C. for 30 minutes. A precipitate of DNA was obtained by centrifuging the solution.

The DNA precipitate was dissolved in 20 μl of TE.

Using 5 μl of the dissolved solution, the DNA was digested with restriction enzymes ClaI and BamHI, the resulting DNA fragments were separated by agarose gel electrophoresis, and then a DNA fragment of 1.2 kb was recovered by using Gene Clean II Kit (purchased from Funakoshi).

After 0.2 μg of pBluescript II SK(+) was digested with restriction enzymes ClaI and BamHI, the resulting DNA fragments were separated by agarose gel electrophoresis, and then a DNA fragment of 2.9 kb was recovered in the same manner.

Using a ligation kit (manufactured by Takara Shuzo), the 1.2 kb and 2.9 kb fragments were subjected to ligation reaction at 16° C. for 16 hours. Escherichia coli NM522 was transformed by using the ligation reaction solution in accordance with the above known method, and the transformant was spread on an LB agar medium [10 g/l Bacto-Tryptone (manufactured by Difco), 10 g/l yeast extract (manufactured by Difco), 5 g/l sodium chloride and 15 μl agarose] containing 50 μg/ml ampicillin, followed by culturing overnight at 30° C.

A plasmid was extracted from the thus formed transformant colonies in accordance with the above known method, the structure of the plasmid was confirmed by restriction enzyme digestion, and the plasmid was named pPMO188SK. The plasmid pPMO188SK has been deposited on Sep. 13, 2001, as FERM BP-7730 in International Patent Organism Depositary, National Institute of Advanced Industrial Science and Technology (Central 6, 1-1, Higashi 1-Chome Tsukuba, Ibaraki, 305-8566, Japan).

Next, pPMO188SK was digested with restriction enzymes ClaI and BamHI, the resulting DNA fragments were separated by agarose gel electrophoresis, and then a DNA fragment of 1.2 kb was recovered. After 0.2 μg of pPAC31 DNA was digested with restriction enzymes ClaI and BamHI, the resulting DNA fragments were separated by agarose gel electrophoresis, and then a DNA fragment of 5.4 kb was recovered in the same manner.

Using the ligation kit, the 1.2 kb and 5.4 kb fragments were subjected to ligation reaction at 16° C. for 16 hours.

Escherichia coli NM522 was transformed by using the ligation reaction solution in accordance with the above known method, and the transformant was spread on the LB agar medium containing 50 μg/ml ampicillin, followed by culturing overnight at 30° C.

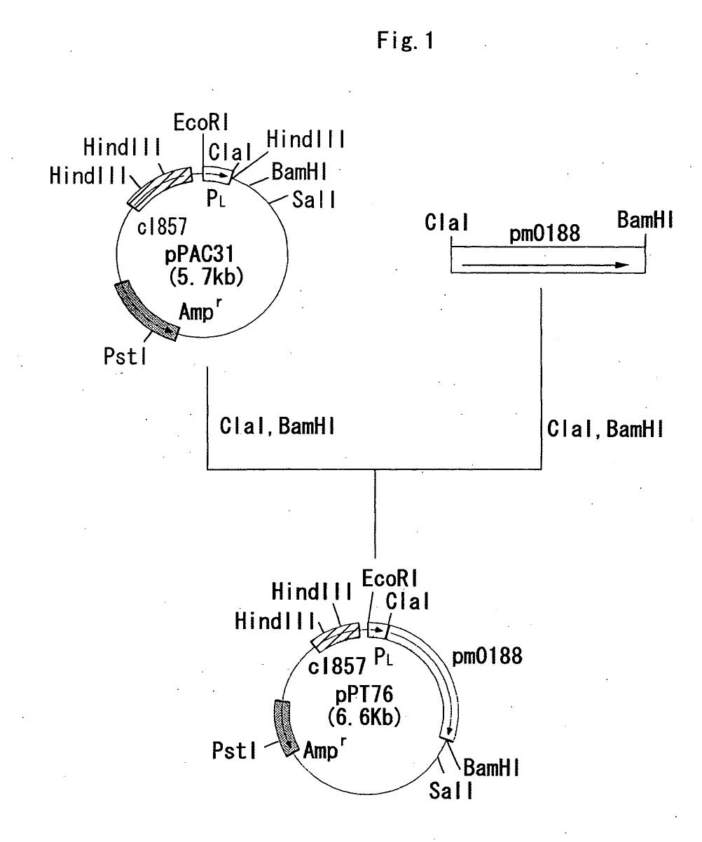

A plasmid was extracted from the thus formed transformant colonies in accordance with the above known method to thereby obtain an expression plasmid pPT76. The structure of the plasmid was confirmed by restriction enzyme digestion (FIG. 1).

(2) Construction of Escherichia coli NM522/pPT79

In the same manner as in the above item (1), a DNA of interest was obtained by PCR in the following manner by using a chromosomal DNA of Pasteurella multocida PM70.

Using DNA fragments having the nucleotide sequences represented by SEQ ID NOs:9 and 10 which had been synthesized by using a DNA synthesizer Model 8905 manufactured by Perceptive Biosystems, a DNA fragment containing pm508 considered to be a gene encoding an α2,3-sialyltransferase in the full nucleotide sequence of the genomic DNA in Pasteurella multocida PM70 was amplified by the following method.

PCR was carried out by using the above synthetic DNA fragments as a primer set and using the chromosomal DNA of Pasteurella multocida PM70 as the template. The PCR was carried out by using 40 μl of a reaction solution containing 0.1 μg of the chromosomal DNA, 0.5 μmol/l of each of the primers, 2.5 units of Pfu DNA polymerase (manufactured by Stratagene), 4 μl of 10×buffer for Pfu DNA polymerase and 200 μmol/l of each deoxy NTP, and repeating 30 times of a step consisting of 1 minute at 94° C., 2 minutes at 42° C. and 3 minutes at 72° C.

A 1/10 volume of the reaction solution was subjected to agarose gel electrophoresis to confirm that the fragment of interest was amplified, and then the remaining reaction solution was mixed with the same volume of TE [10 mmol/l Tris-HCl and 1 mmol/l EDTA (pH 8.0)] saturated phenol/chloroform (1 vol/1 vol).

After the mixed solution was centrifuged, the thus obtained upper layer was mixed with 2 volumes of cold ethanol and allowed to stand at −80° C. for 30 minutes. A precipitate of DNA was obtained by centrifuging the solution,.

The DNA precipitate was dissolved in 20 μl of TE.

Using 5 μl of the dissolved solution, the DNA was digested with restriction enzymes ClaI and BamHI, the resulting DNA fragments were separated by agarose gel electrophoresis, and then a DNA fragment of 1.0 kb was recovered by using the Gene Clean II Kit.

After 0.2 μg of pBluescript II SK(+) was digested with restriction enzymes ClaI and BamHI, the resulting DNA fragments were separated by agarose gel electrophoresis, and then a DNA fragment of 2.9 kb was recovered in the same manner.

Using the ligation kit, the 1.0 kb and 2.9 kb fragments were subjected to ligation reaction at 16° C. for 16 hours. Escherichia coli NM522 was transformed by using the ligation reaction solution in accordance with the above known method, and the transformant was spread on the LB agar medium containing 50 μg/ml ampicillin, followed by culturing overnight at 30° C.

A plasmid was extracted from the thus formed transformant colonies in accordance with the above known method, the structure of the plasmid was confirmed by restriction enzyme digestion, and the plasmid was named pPMO508SK. The plasmid pPMO508SK has been deposited on Sep. 13, 2001, as FERM BP-7731 in International Patent Organism Depositary, National Institute of Advanced Industrial Science and Technology (Central 6, 1-1, Higashi 1-Chome Tsukuba, Ibaraki, 305-8566, Japan).

Next, pPMO508SK was digested with restriction enzymes ClaI and BamHI, the resulting DNA fragments were separated by agarose gel electrophoresis, and then a DNA fragment of 1.0 kb was recovered. After 0.2 μg of pPAC31 DNA was digested with restriction enzymes ClaI and BamHI, the resulting DNA fragments were separated by agarose gel electrophoresis, and then a DNA fragment of 5.4 kb was recovered in the same manner.

Using the ligation kit, the 1.0 kb and 5.4 kb fragments were subjected to ligation reaction at 16° C. for 16 hours.

Escherichia coli NM522 was transformed by using the ligation reaction solution in accordance with the above known method, and the transformant was spread on the LB agar medium containing 50 μg/ml ampicillin, followed by culturing overnight at 30° C.

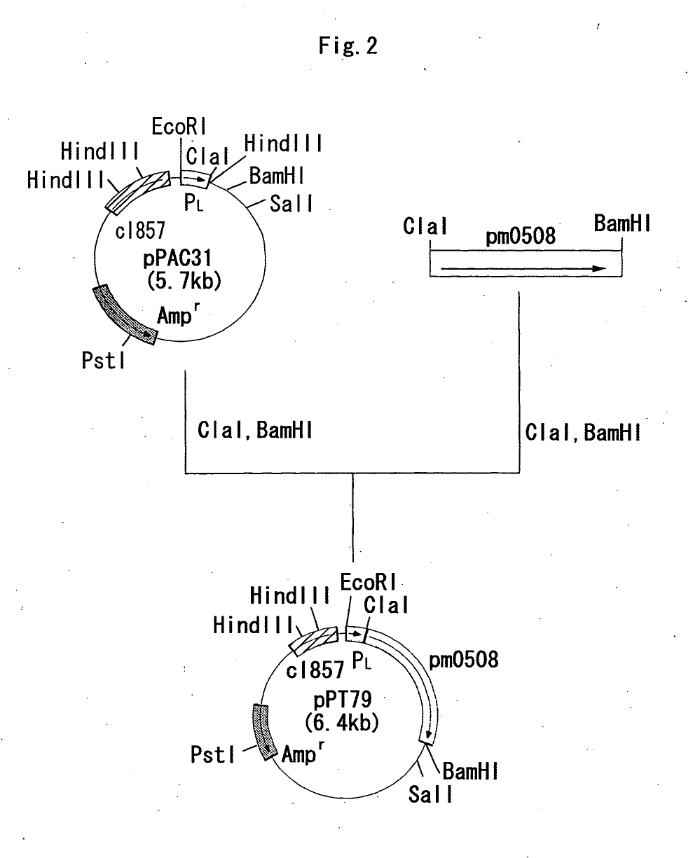

A plasmid was extracted from the thus formed transformant colonies in accordance with the above known method to thereby obtain an expression plasmid pPT79. The structure of the plasmid was confirmed by restriction enzyme digestion (FIG. 2).

(3) Construction of Escherichia coli NM522/pPT78

In the same manner as in the above item (1), a DNA of interest was obtained by PCR using chromosomal DNA of Pasteurella multocida PM70.

Using DNA fragments having the nucleotide sequences represented by SEQ ID NOs:11 and 12 which had been synthesized by using a DNA synthesizer Model 8905 manufactured by Perceptive Biosystems, a DNA fragment containing pm1174 considered to be a gene encoding an α2,3-sialyltransferase in the full nucleotide sequence of the genomic DNA in Pasteurella multocida PM70 was amplified by the following method.

PCR was carried out by using the above synthetic DNA fragments as a primer set and using the chromosomal DNA of Pasteurella multocida PM70 as the template. The PCR was carried out by using 40 μl of a reaction solution containing 0.1 μg of the chromosomal DNA, 0.5 μmol/l of each of the primers, 2.5 units of Pfu DNA polymerase (manufactured by Stratagene), 4 μl of 10×buffer for Pfu DNA polymerase and 200 μmol/l of each deoxy NTP, and repeating 30 times of a step consisting of 1 minute at 94° C., 2 minutes at 42° C. and 3 minutes at 72° C.

A 1/10 volume of the reaction solution was subjected to agarose gel electrophoresis to confirm that the fragment of interest was amplified, and then the remaining reaction solution was mixed with the same volume of TE [10 mmol/l Tris-HCl and 1 mmol/l EDTA (pH 8.0)] saturated phenol/chloroform (1 vol/1 vol).

After centrifugation of the mixed solution, the thus obtained upper layer was mixed with 2 volumes of cold ethanol and allowed to stand at −80° C. for 30 minutes. A precipitate of DNA was obtained by centrifuging the solution.

The DNA precipitate was dissolved in 20 μl of TE.

Using 5 μl of the dissolved solution, the DNA was digested with restriction enzymes ClaI and BamHI, the resulting DNA fragments were separated by agarose gel electrophoresis, and then a DNA fragment of 0.9 kb was recovered by using Gene Clean II Kit (purchased from Funakoshi).

After 0.2 μg of pBluescript II SK(+) was digested with restriction enzymes ClaI and BamHI, the resulting DNA fragments were separated by agarose gel electrophoresis, and then a DNA fragment of 2.9 kb was recovered in the same manner.

Using the ligation kit, the 0.9 kb and 2.9 kb fragments were subjected to ligation reaction at 16° C. for 16 hours. Escherichia coli NM522 was transformed by using the ligation reaction solution in accordance with the above known method, and the transformant was spread on the LB agar medium containing 50 μg/ml ampicillin, followed by culturing overnight at 30° C.

A plasmid was extracted from the thus formed transformant colonies in accordance with the above known method, the structure of the plasmid was confirmed by restriction enzyme digestion, and the plasmid was named pPM1174SK. The plasmid pPM1174SK has been deposited on Sep. 13, 2001, as FERM BP-7733 in International Patent Organism Depositary, National Institute of Advanced Industrial Science and Technology (Central 6, 1-1, Higashi 1-Chome Tsukuba, Ibaraki, 305-8566, Japan).

Next, pPM1174SK was digested with restriction enzymes ClaI and BamHI, the resulting DNA fragments were separated by agarose gel electrophoresis, and then a DNA fragment of 0.9 kb was recovered. After 0.2 μg of pPAC31 DNA was digested with restriction enzymes ClaI and BamHI, the resulting DNA fragments were separated by agarose gel electrophoresis, and then a DNA fragment of 5.4 kb was recovered in the same manner.

Using the ligation kit, the 0.9 kb and 5.4 kb fragments were subjected to ligation reaction at 16° C. for 16 hours.

Escherichia coli NM522 was transformed by using the ligation reaction solution in accordance with the above known method, and the transformant was spread on the LB agar medium containing 50 μg/ml ampicillin, followed by culturing overnight at 30° C.

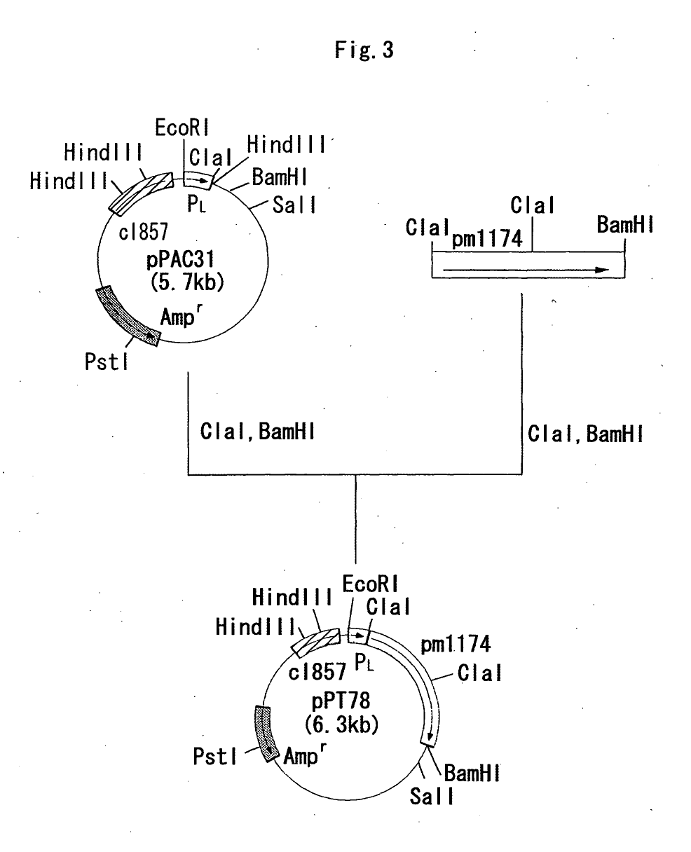

A plasmid was extracted from the thus formed transformant colonies in accordance with the above known method to thereby obtain an expression plasmid pPT78. The structure of the plasmid was confirmed by restriction enzyme digestion (FIG. 3).

### EXAMPLE 2

### Production of a Sialic Acid-Containing Complex Carbohydrates Using Escherichia Coli NM522/pPT76

(1) Production of NeuAcα2-3Galβ1-4Glc

Escherichia coli NM522/pPT76 obtained in the item (1) of Example 1 was inoculated into a test tube charged with 8 ml of LB medium containing 50 μg/ml ampicillin, followed by culturing at 28° C. for 17 hours. The culture was inoculated into a test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 16 hours in 0.1 ml of a reaction solution containing the NM522/pPT76 wet cells obtained in the above, 50 mmol/l citrate buffer (pH 7.0), 5 mmol/l MnCl₂, 10 mmol/l lactose, 5 mmol/l CMP-sialic acid and 4 μl Nymeen S-215.

After completion of the reaction, the reaction product was analyzed by using a sugar analyzer manufactured by Dionex (DX-500) under the following analyzing conditions to confirm that 2.69 mmol/l (1,704 mg/l) NeuAcα2-3Galβ1-4Glc was formed and accumulated in the reaction solution.

Analyzing Conditions:

Column: CarboPAC PA10 (manufactured by Dionex)
Eluent: eluent A; 500 mmol/l NaOH, eluent B; 100 mmol/l NaOH+500 mmol/l NaOAc
Gradient: Linear gradient from a composition comprising 20% of the eluent A and 10% of the eluent B at 0 minute to a composition comprising 30% of the eluent A and 25% of the eluent B spending 30 minutes.
Detector: Pulsed amperometry detector.
   (2) Production of NeuAcα2-3Galβ1-4GlcNAcβ1-3Galβ1-4Glc

Escherichia coli NM522/pPT76 obtained in the item (1) of Example 1(1) was inoculated into a test tube charged with 8 ml of LB medium containing 50 μg/ml ampicillin, followed by culturing at 28° C. for 17 hours. The culture was inoculated into a test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 16 hours in 0.1 ml of a reaction solution containing the NM522/pPT76 wet cells obtained in the above, 50 mmol/l citrate buffer (pH 7.0), 5 mmol/l MnCl₂, 10 mmol/l lacto-N-neotetraose, 5 mmol/l CMP-sialic acid and 4 g/l Nymeen S-215.

After completion of the reaction, the reaction product was analyzed by using a sugar analyzer manufactured by Dionex (DX-500) under the analyzing conditions described in the item (1) of Example 2 to confirm that 1.64 mmol/l (1,638 mg/l) NeuAcα2-3Galβ1-4GlcNacβ1-3Galβ1-4Glc was formed and accumulated in the reaction solution.

(3) Production of NeuAcα2-8NeuAcα2-3Galβ1-4Glc

Escherichia coli NM522/pPT76 obtained in the item (1) of Example 1 was inoculated into a thick test tube charged with 8 ml of LB medium containing 50 μg/ml ampicillin, followed by culturing at 28° C. for 17 hours. The culture was inoculated into a thick test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 2 hours in 0.1 ml of a reaction solution containing the NM522/pPT76 wet cells obtained in the above, 50 mmol/l citrate buffer (pH 7.0), 5 mmol/l MnCl₂, 2 mmol/l NeuAcα2-3Galβ1-4Glc, 2 mmol/l CMP-sialic acid and 4 g/l Nymeen S-215.

After completion of the reaction, the reaction product was analyzed by using a sugar analyzer manufactured by Dionex (DX-500) under the following analyzing conditions to confirm that 0.43 mmol/l (397 mg/l) NeuAcα2-8NeuAcα2-3Galβ1-4Glc was formed and accumulated in the reaction solution.

Analyzing conditions:

Column: CarboPAC PA10
Eluent: eluent A; H₂O, eluent B; 500 mmol/l NaOH, eluent C; 100 mmol/l NaOH+500 mmol/l NaOAc
Gradient: Linear gradient from a composition comprising 10% of the eluent A, 60% of the eluent B and 30% of the eluent C at 0 minute to a composition comprising 0% of the eluent A, 65% of the eluent B and 35% of the eluent C spending 15 minutes.
Detector: Pulsed amperometry detector.

### EXAMPLE 3

### Production of Sialic Acid-Containing Complex Carbohydrates Using Escherichia Coli NM522/pPT79

(1) Production of NeuAcα2-3Galβ1-4Glc

Escherichia coli NM522/pPT79 obtained in the item (2) of Example 1 was inoculated into a test tube charged with 8 ml of LB medium containing 50 μg/ml ampicillin, followed by culturing at 28° C. for 17 hours. The culture was inoculated into a test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 6 hours in 0.1 ml of a reaction solution containing the NM522/pPT79 wet cells obtained in the above, 50 mmol/l citrate buffer (pH 7.0), 5 mmol/l MnCl₂, 10 mmol/l lactose, 5 mmol/l CMP-sialic acid and 4 g/l Nymeen S-215.

After completion of the reaction, the reaction product was analyzed by using a sugar analyzer manufactured by Dionex (DX-500) under the analyzing conditions described in the item (1) of Example 2 to confirm that 0.25 mmol/l (164 mg/l) NeuAcα2-3Galβ1-4Glc was formed and accumulated in the reaction solution.

(2) Production of NeuAcα2-8NeuAcα2-3Galβ1-4Glc

Escherichia coli NM522/pPT79 obtained in the item (2) of Example 1 was inoculated into a thick test tube charged with 8 ml of LB medium containing 50 μg/ml ampicillin, followed by culturing at 28° C. for 17 hours. The culture was inoculated into a thick test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 2 hours in 0.1 ml of a reaction solution containing the NM522/pPT79 wet cells obtained in the above, 50 mmol/l citrate buffer (pH 7.0), 5 mmol/l MnCl₂, 2 mmol/l NeuAcα2-3Galβ1-4Glc, 2 mmol/l CMP-sialic acid and 4 g/l Nymeen S-215.

After completion of the reaction, the reaction product was analyzed by using a sugar analyzer manufactured by Dionex (DX-500) under the analyzing conditions described in the item (3) of Example 2 to confirm that 0.54 mmol/l (499 mg/l) NeuAcα2-8NeuAcα2-3Galβ1-4Glc was formed and accumulated in the reaction solution.

### EXAMPLE 4

### Production of Sialic Acid-Containing Complex Carbohydrates Using Escherichia coli NM522/pPT78

(1) Production of NeuAcα2-3Galβ1-4Glc

Escherichia coli NM522/pPT78 obtained in the item (3) of Example 1 was inoculated into a test tube charged with 8 ml of LB medium containing 50 μg/ml ampicillin, followed by culturing at 28° C. for 17 hours. The culture was inoculated into a test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 16 hours in 0.1 ml of a reaction solution containing the NM522/pPT78 wet cells obtained in the above, 50 mmol/l citrate buffer (pH 7.0), 5 mmol/l MnCl₂, 10 mmol/l lactose, 5 mmol/l CMP-sialic acid and 4 g/l Nymeen S-215.

After completion of the reaction, the reaction product was analyzed using a sugar analyzer manufactured by Dionex (DX-500) under the analyzing conditions described in the item (1) of Example 2 to confirm that 0.98 mmol/l (621 mg/l) NeuAcα2-3Galβ1-4Glc was formed and accumulated in the reaction solution.

(2) Production of NeuAcα2-8NeuAcα2-3Galβ1-4Glc

Escherichia coli NM522/pPT78 obtained in the item (3) of Example 1 was inoculated into a test tube charged with 8 ml of LB medium containing 50 μg/ml ampicillin, followed by culturing at 28° C. for 17 hours. The culture was inoculated into a test tube charged with 8 ml of LB medium containing 50 μg/ml ampicillin, with an inoculum size of 1%, followed by culturing at 28° C. for 2 hours and then at 40° C. for 3 hours. Wet cells were obtained by centrifuging 0.5 ml of the culture. The wet cells can be stored at −20° C., if necessary, and it was able to use them by thawing prior to use.

The reaction was carried out at 37° C. for 2 hours in 0.1 ml of a reaction solution containing the NM522/pPT78 wet cells obtained in the above, 50 mmol/l citrate buffer (pH 7.0), 5 mmol/l MnCl₂, 2 mmol/l NeuAcα2-3Galβ1-4Glc, 2 mmol/l CMP-sialic acid and 4 g/l Nymeen S-215.

After completion of the reaction, the reaction product was analyzed by using a sugar analyzer manufactured by Dionex (DX-500) under the analyzing conditions described in the item (3) of Example 2 to confirm that 0.43 mmol/l (397 mg/l) NeuAcα2-8NeuAcα2-3Galβ1-4Glc was formed and accumulated in the reaction solution.

### INDUSTRIAL APPLICABILITY

According to the present invention, an α2,3/α2,8-sialyltransferase can be produced in a large amount. Also, a sialic acid-containing complex carbohydrate can be produced efficiently by using the enzyme.

Free Text of Sequence Listing:

SEQ ID NO:7—Description of artificial sequence: Synthetic DNA
SEQ ID NO:8—Description of artificial sequence: Synthetic DNA
SEQ ID NO:9—Description of artificial sequence: Synthetic DNA
SEQ ID NO:10—Description of artificial sequence: Synthetic DNA.
SEQ ID NO:11—Description of artificial sequence: Synthetic DNA
SEQ ID NO:12—Description of artificial sequence: Synthetic DNA

1. A process for producing a protein having α2,3/α2,8-sialyltransferase activity, which comprises: culturing a transformant capable of producing a protein having α2,3/α2,8-sialyltransferase activity derived from a microorganism belonging to the genus Pasteurella in a medium to produce and accumulate the protein having α2,3/α2,8-sialyltransferase activity in the culture, and recovering the protein from the culture.

2. The process according to claim 1, wherein the microorganism belonging to the genus Pasteurella is Pasteurella multocida.

3. The process according to claim 1, wherein the protein having α2,3/α2,8-sialyltransferase activity is a protein comprising the amino acid sequence represented by any one selected from SEQ ID NOs:1, 3 and 5.

4. The process according to claim 1, wherein the protein having α2,3/α2,8-sialyltransferase activity is a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity.

5. The process according to claim 1, wherein the protein having α2,3/α2,8-sialyltransferase activity is a protein which has an identity of at least 80% with the amino acid sequence represented by any one selected from SEQ ID NOs:1, 3 and 5, and has α2,3/α2,8-sialyltransferase activity.

6. The process according to claim 1, wherein the transformant is a transformant which comprises a recombinant DNA comprising a DNA encoding a protein having α2;3/α2,8-sialyltransferase activity.

7. The process according to claim 6, wherein the transformant is obtained by using a microorganism as a host cell.

8. The process according to claim 7, wherein the microorganism is Escherichia coli.

9. The process according to claim 6, wherein the DNA encoding the protein having α2,3/α2,8-sialyltransferase activity is a DNA described in one of the following (a) to (e):
(a) a DNA encoding a protein comprising the amino acid sequence selected from one of SEQ ID NOs:1, 3 and 5;
(b) a DNA encoding a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity;
(c) a DNA encoding a protein which has an identity of at least 80% with the amino acid sequence represented by any one selected from SEQ ID NOs:1, 3 and 5, and has α2,3/α2,8-sialyltransferase activity;
(d) a DNA comprising the nucleotide sequence represented by any one selected from SEQ ID NOs:2, 4 and 6; and
(e) a DNA which hybridizes with a DNA consisting of a complementary sequence of the nucleotide sequence represented by any one selected from SEQ ID NOs:2, 4 and 6 under stringent conditions, and encodes a protein having α2,3/α2,8-sialyltransferase activity.

10. A process for producing a sialic acid-containing complex carbohydrate, which comprises: allowing a culture of a transformant capable of producing a protein having α2,3/α2,8-sialyltransferase activity derived from a microorganism or a treated product of the culture as an enzyme source, cytidine-5′-monophosphosialic acid and an acceptor complex carbohydrate to be present in an aqueous medium to produce and accumulate the sialic acid-containing complex carbohydrate in the aqueous medium, and recovering the sialic acid-containing complex carbohydrate from the aqueous medium.

11. The process according to claim 10, wherein the treated product of the culture is selected from the group consisting of a concentrated product of the culture, a dried product of the culture, cells obtained by centrifuging the culture, a dried product of the cells, a freeze-dried product of the cells, a surfactant-treated product of the cells, an ultrasonic-treated product of the cells, a mechanically disrupted product of the cells, a solvent-treated product of the cells, an enzyme-treated product of the cells, a protein fraction of the cells, an immobilized product of the cells and an enzyme preparation obtained by extracting from the cells.

12. The process according to claim 10, wherein the acceptor complex carbohydrate is a complex carbohydrate comprising an oligosaccharide having galactose in its non-reducing terminal.

13. The process according to claim 10, wherein the acceptor complex carbohydrate is a complex carbohydrate comprising an oligosaccharide having sialic acid in its non-reducing terminal.

14. The process according to claim 12, wherein the oligosaccharide having galactose in its non-reducing terminal is an oligosaccharide selected from the group consisting of lactose, globotriose, N-acetyllactosamine, lacto-N-tetraose, lacto-N-neotetraose, Lewis^{a} and Lewis^{X}.

15. The process according to claim 13, wherein the oligosaccharide having sialic acid in its non-reducing terminal is an oligosaccharide selected from the group consisting of NeuAcα2-3Galβ1-4Glc and NeuAcα2-3Galβ1-4GlcNAc.

16. The process according to claim 10, wherein the microorganism is a microorganism belonging to the genus Pasteurella.

17. The process according to claim 16, wherein the microorganism belonging to the genus Pasteurella is Pasteurella multocida.

18. The process according to claim 10, wherein the protein having α2,3/α2,8-sialyltransferase activity is a protein comprising the amino acid sequence represented by any one selected from SEQ ID NOs:1, 3 and 5.

19. The process according to claim 10, wherein the protein having α2,3/α2,8-sialyltransferase activity is a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity.

20. The process according to claim 10, wherein the protein having α2,3/α2,8-sialyltransferase activity is a protein which has an identity of at least 80% with the amino acid sequence represented by any one selected from SEQ ID NOs:1, 3 and 5, and has α2,3/α2,8-sialyltransferase activity.

21. The process according to claim 10, wherein the transformant is a transformant which comprises a recombinant DNA comprising a DNA encoding a protein having α2,3/α2,8-sialyltransferase activity.

22. The process according to claim 21, wherein the transformant is obtained by using a microorganism as a host cell.

23. The process according to claim 22, wherein the microorganism is Escherichia coli.

24. The process according to claim 21, wherein the DNA encoding the protein having α2,3/α2,8-sialyltransferase activity is a DNA represented by one of the following (a) to (e):
(a) a DNA encoding a protein comprising the amino acid sequence selected from one of SEQ ID NOs:1, 3 and 5;
(b) a DNA encoding a protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity;
(c) a DNA encoding a protein which has an identity of at least 80% with the amino acid sequence represented by any one selected from SEQ ID NOs:1, 3 and 5, and has α2,3/α2,8-sialyltransferase activity;
(d) a DNA comprising the nucleotide sequence represented by any one selected from SEQ ID NOs:2, 4 and 6; and
(e) a DNA which hybridizes with a DNA consisting of a complementary sequence of the nucleotide sequence represented by any one selected from SEQ ID NOs:2, 4 and 6 under stringent conditions, and encodes a protein having α2,3/α2,8-sialyltransferase activity.

25. A protein having α2,3/α2,8-sialyltransferase activity, which comprises the amino acid sequence represented by SEQ ID NO:1.

26. A protein having α2,3/α2,8-sialyltransferase activity, which comprises the amino acid sequence represented by SEQ ID NO:3.

27. A protein having α2,3/α2,8-sialyltransferase activity, which comprises the amino acid sequence represented by SEQ ID NO:5.

28. A protein which consists of an amino acid sequence in which at least one amino acid is deleted, substituted or added in the amino acid sequence represented by any one selected from SEQ ID NOs:1, 3 and 5, and has α2,3/α2,8-sialyltransferase activity.

29. A protein which has an identity of at least 80% with the amino acid sequence represented by any one selected from SEQ ID NOs:1, 3 and 5, and has α2,3/α2,8-sialyltransferase activity.
